(19) United States
(12) Patent Application Publication
(10) Pub. No.: US 2021/0345923 A1
(43) Pub. Date: Nov. 11, 2021

(54) REDUNDANT EYE TRACKING SYSTEM

(71) Applicant: Tobii AB, Danderyd (SE)

(72) Inventors: Per Fogelström, Danderyd (SE); Anna Redz, Danderyd (SE); Anders Dahl, Danderyd (SE); Niklas Ollesson, Danderyd (SE)

(73) Assignee: Tobii AB, Danderyd (SE)

(21) Appl. No.: 17/236,846

(22) Filed: Apr. 21, 2021

(30) Foreign Application Priority Data
Apr. 21, 2020 (SE) 2030135-4

(51) Int. Cl. A61B 5/16; A61B 5/18

(52) U.S. Cl. CPC A61B 5/163; A61B 5/18

(57) ABSTRACT

There is provided mechanisms for eye position determination of a subject depicted in an image set. A method comprises obtaining first information indicating a first set of eye positions of the subject by applying a first eye tracking procedure on an image set depicting the subject. The first eye tracking procedure uses a first set of features extracted from the image set for obtaining the first set of eye positions. The method comprises obtaining second information indicating a second set of eye positions of the subject by applying a second eye tracking procedure on the image set depicting the subject. The second eye tracking procedure uses a second set of features extracted from the image set for obtaining the second set of eye positions. The method comprises determining the eye position of the subject in the image set based on the first information and the second information only when the first set of eye positions, as indicated by the first information, and the second set of eye positions, as indicated by the second information, differ from each other less than a threshold value.

### CROSS-REFERENCE TO RELATED APPLICATION

The present application claims benefit to Swedish patent application No 2030135-4, filed Apr. 21, 2020, entitled “REDUNDANT EYE TRACKING SYSTEM”, and is hereby incorporated by reference in its entirety.

### TECHNICAL FIELD

Embodiments presented herein relate to a method, an eye tracking system, a computer program, and a computer program product for eye position determination of a subject.

### BACKGROUND

Eye tracking is a sensor technology that makes it possible for a computer or other device to know where a subject, such as a person, is looking. An eye tracker can detect the presence, attention and focus of the user. Eye tracking need not necessarily involve tracking of the user's gaze (for example in the form of a gaze direction or a gaze point). Eye tracking may for example relate to tracking of the position of an eye of the subject in space, without actually tracking a gaze direction or gaze point of the eye.

Different techniques have been developed for monitoring in which direction (or at which point on a display) a subject, such as person, is looking. This is often referred to as gaze tracking. Such techniques often involve detection of certain features in images of the eye, and a gaze direction or gaze point is then computed based on positions of these detected features. An example of such a gaze tracking technique is pupil center corneal reflection (PCCR). PCCR based gaze tracking employs the position of the pupil center and the position of glints (reflections of illuminators at the cornea) to compute a gaze direction of the eye or a gaze point at a display.

As an alternative (or complement) to conventional techniques such as PCCR-based eye tracking, machine learning may be employed to train an algorithm to perform eye tracking. For example, the machine learning may employ training data in the form of images of the eye and associated known gaze points to train the algorithm, so that the trained algorithm can perform eye tracking in real time based on images of the eye.

Plenty of training data is typically needed for such machine learning to work properly. The training data may take quite some time and/or resources to collect. In many cases, certain requirements may be put on the training data. The training data should for example preferably reflect all those types of cases/scenarios that the eye tracking algorithm is supposed to be able to handle. If only certain types of cases/scenarios are represented in the training data (for example only small gaze angles, or only well-illuminated images), then the eye tracking algorithm may perform well for such cases/scenarios, but may not perform that well for other cases/scenarios not dealt with during the training phase.

Hence, each type of eye tracker comes with its own advantages and disadvantages. However, regardless of which type of eye tracker is used, there is a risk that the performance of the eye tracker will be inaccurate, for example due to software or hardware issues.

It would be desirable to provide new ways to address one or more of the abovementioned issues.

### SUMMARY

An object of embodiments herein is to address one or more of the issues noted above.

According to a first aspect there is presented a method for eye position determination of a subject depicted in an image set. The method comprises obtaining first information indicating a first set of eye positions of the subject by applying a first eye tracking procedure on an image set depicting the subject. The first eye tracking procedure uses a first set of features extracted from the image set for obtaining the first set of eye positions. The method comprises obtaining second information indicating a second set of eye positions of the subject by applying a second eye tracking procedure on the image set depicting the subject. The second eye tracking procedure uses a second set of features extracted from the image set for obtaining the second set of eye positions. The method comprises determining the eye position of the subject in the image set based on the first information and the second information only when the first set of eye positions, as indicated by the first information, and the second set of eye positions, as indicated by the second information, differ from each other less than a threshold value.

According to a second aspect there is presented an eye tracking system for eye position determination of a subject depicted in an image set. The eye tracking system is configured to obtain first information indicating a first set of eye positions of the subject by applying a first eye tracking procedure on an image set depicting the subject. The first eye tracking procedure uses a first set of features extracted from the image set for obtaining the first set of eye positions. The eye tracking system is configured to obtain second information indicating a second set of eye positions of the subject by applying a second eye tracking procedure on the image set depicting the subject. The second eye tracking procedure uses a second set of features extracted from the image set for obtaining the second set of eye positions. The eye tracking system is configured to determine the eye position of the subject in the image set based on the first information and the second information only when the first set of eye positions, as indicated by the first information, and the second set of eye positions, as indicated by the second information, differ from each other less than a threshold value.

According to a third aspect there is presented an eye tracking system for eye position determination of a subject depicted in an image set. The eye tracking system comprises an obtain module configured to obtain first information indicating a first set of eye positions of the subject by applying a first eye tracking procedure on an image set depicting the subject. The first eye tracking procedure uses a first set of features extracted from the image set for obtaining the first set of eye positions. The eye tracking system comprises an obtain module configured to obtain second information indicating a second set of eye positions of the subject by applying a second eye tracking procedure on the image set depicting the subject. The second eye tracking procedure uses a second set of features extracted from the image set for obtaining the second set of eye positions. The eye tracking system comprises a determine module configured to determine the eye position of the subject in the image set based on the first information and the second information only when the first set of eye positions, as indicated by the first information, and the second set of eye positions, as indicated by the second information, differ from each other less than a threshold value,

According to a fourth aspect there is presented a computer program for eye position determination of a subject depicted in an image set, the computer program comprising computer program code which, when run on an eye tracking system, causes the eye tracking system to perform a method according to the first aspect.

According to a fifth aspect there is presented a computer program product comprising a computer program according to the fourth aspect and a computer readable storage medium on which the computer program is stored. The computer readable storage medium could be a non-transitory computer readable storage medium.

Advantageously these aspects provide efficient determination of the eye position of the subject in the image set.

Advantageously these aspects provide a redundant eye tracking system.

Other objectives, features and advantages of the enclosed embodiments will be apparent from the following detailed disclosure, from the attached dependent claims as well as from the drawings.

Generally, all terms used in the claims are to be interpreted according to their ordinary meaning in the technical field, unless explicitly defined otherwise herein. All references to “a/an/the element, apparatus, component, means, module, step, etc.” are to be interpreted openly as referring to at least one instance of the element, apparatus, component, means, module, step, etc., unless explicitly stated otherwise. The steps of any method disclosed herein do not have to be performed in the exact order disclosed, unless explicitly stated.

### BRIEF DESCRIPTION OF THE DRAWINGS

The inventive concept is now described, by way of example, with reference to the accompanying drawings, in which:

FIG. 1 is a front view of an eye;

FIG. 2 is a cross sectional view of the eye from FIG. 1 from the side of the eye;

FIG. 3 schematically illustrates a face model as applied to a subject;

FIG. 4 is a flowchart of methods according to embodiments;

FIG. 5 is a schematic diagram of a redundant eye tracking system according to an embodiment;

FIG. 6 schematically illustrates relation between eye position, gaze origin, gaze direction and gaze point;

FIG. 7 is a schematic diagram showing functional units of an eye tracking system according to an embodiment;

FIG. 8 is a schematic diagram showing functional modules of an eye tracking system according to an embodiment; and

FIG. 9 shows one example of a computer program product comprising computer readable storage medium according to an embodiment.

### DETAILED DESCRIPTION

The inventive concept will now be described more fully hereinafter with reference to the accompanying drawings, in which certain embodiments of the inventive concept are shown. This inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like elements throughout the description. Any step or feature illustrated by dashed lines should be regarded as optional.

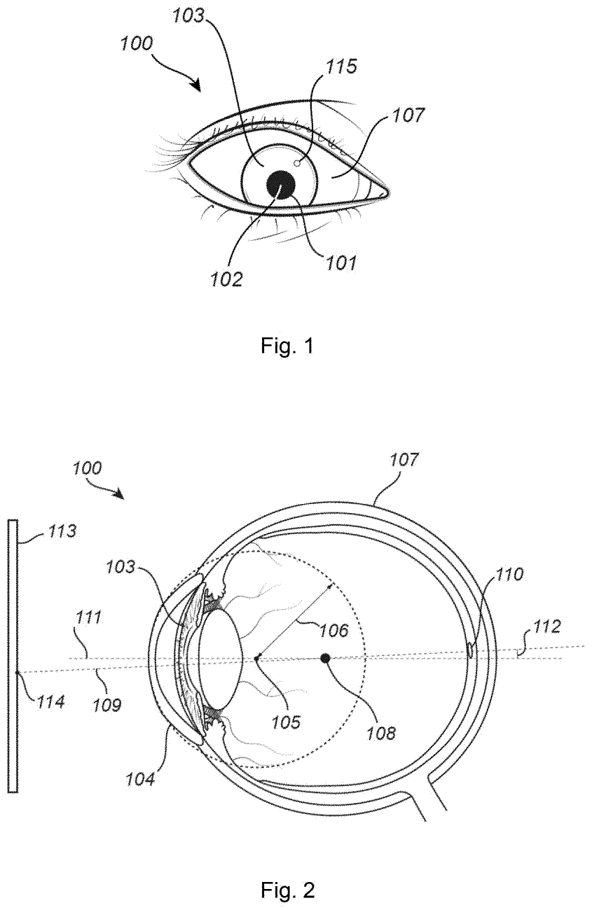

Certain features of an eye will be described with parallel references to FIG. 1 and FIG. 2. FIG. 1 is a front view of an eye 100. FIG. 2 is a cross sectional view of the eye 100 from the side of the eye 100. While FIG. 2 shows more or less the entire eye 100, the front view presented in FIG. 1 only shows those parts of the eye 100 which are typically visible from in front of a person's face. The eye 100 has a pupil 101, which has a pupil center 102. The eye 100 also has an iris 103 and a cornea 104. The cornea 104 is located in front of the pupil 101 and the iris 103. The cornea 104 is curved and has a center of curvature 105 which is referred to as the center 105 of corneal curvature, or simply the cornea center 105. The cornea 104 has a radius of curvature 106 referred to as the radius 106 of the cornea 104, or simply the cornea radius 106.

The eye 100 also has a sclera 107. The eye 100 has a center 108 which may also be referred to as the center 108 of the eye ball, or simply the eye ball center 108. The visual axis 109 of the eye 100 passes through the center 108 of the eye 100 to the fovea 110 of the eye 100. The optical axis 111 of the eye 100 passes through the pupil center 102 and the center 108 of the eye 100. The visual axis 109 forms an angle 112 relative to the optical axis 111. The deviation or offset between the visual axis 109 and the optical axis 111 is often referred to as the fovea offset 112. In the example shown in FIG. 2, the eye 100 is looking towards a display 113, and the eye 100 is gazing at a gaze point 114 at the display 113. FIG. 1 also shows a reflection 115 of an illuminator at the cornea 104. Such a reflection 115 is also known as a glint 115.

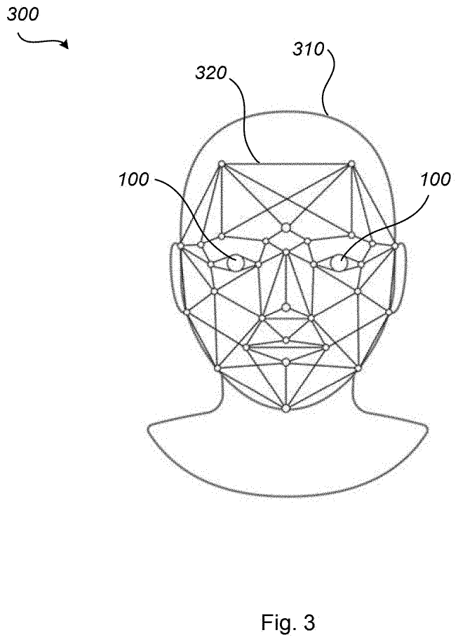

FIG. 3 at 300 schematically illustrates a face model 320 (provided as a polygonal face model) as applied to a subject 310 and where the face model 320 is matched to the positions of the eyes 100 of the subject 310. Such a face model 320 can be used by a non-PCCR based eye tracking procedure.

Issues with different types of known eye trackers have been noted above. Further considerations relating hereto will be disclosed next.

Regardless of what type of eye tracker is used, there is a risk that the eye tracker will suffer from hardware and/or software issues that might negatively impact the end result (i.e., the output produced by the eye tracker) even though the eye tracker is calibrated. Having a redundant eye tracking system running multiple copies of one and the same eye tracking procedure on the same input might reduce the risk of hardware and/or software issues to some degree, but there is still a risk that the multiple copies of one and the same eye tracking procedure will all experience the same issues and hence in this respect only provide a false sense of added redundancy.

The embodiments disclosed herein therefore relate to mechanisms for eye position determination of a subject 310 depicted in an image set, for example using an eye tracking system with redundant eye tracker modules. In order to obtain such mechanisms there is provided an eye tracking system 500, a method performed by the eye tracking system 500, a computer program product comprising code, for example in the form of a computer program, that when run on an eye tracking system 500, causes the eye tracking system 500 to perform the method.

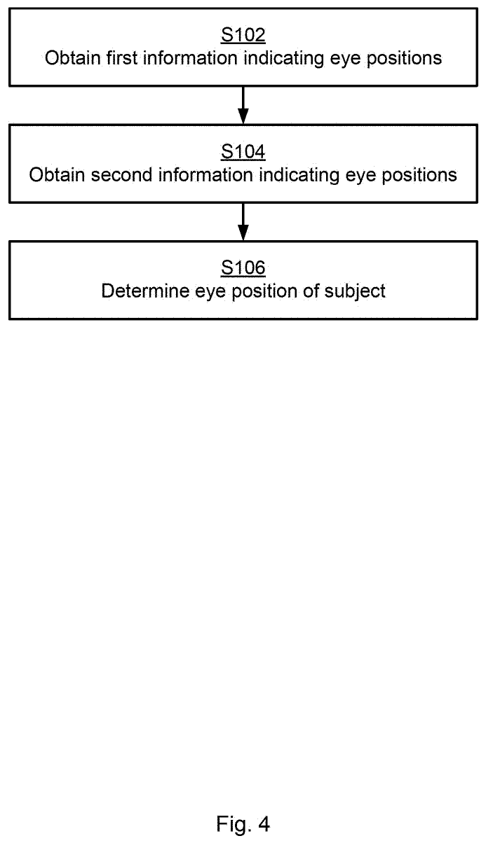

FIG. 4 is a flowchart illustrating embodiments of methods for eye position determination of a subject 310 depicted in an image set. The methods are performed by the eye tracking system 500. The methods are advantageously provided as computer programs 920.

At least some of the herein disclosed embodiments are based on using a redundant eye tracking system 500 running two (independent) eye tracking procedures on the same input image set and where the eye positions are determined only when these eye tracking procedures yield similar results.

S102: First information indicating a first set of eye positions 100 of the subject 310 is obtained by applying a first eye tracking procedure on an image set 510 depicting the subject 310. The first eye tracking procedure uses a first set of features extracted from the image set 510 for obtaining the first set of eye positions 100.

S104: Second information indicating a second set of eye positions 100 of the subject 310 is obtained by applying a second eye tracking procedure on the image set 510 depicting the subject 310. The second eye tracking procedure uses a second set of features extracted from the image set 510 for obtaining the second set of eye positions 100.

The output produced by the first eye tracking procedure and the second eye tracking procedure might thus be compared to each other.

In some examples the image set 510 comprises a sequence of images depicting the subject 310. In other aspects the image set 510 comprises a single image of the subject 310. The image set 510 might thus either be composed of a sequence of digital image frames or a single such digital image frame.

S106: The eye position of the subject 310 in the image set 510 is determined based on the first information and the second information only when the first set of eye positions 100, as indicated by the first information, and the second set of eye positions 100, as indicated by the second information, differ from each other less than a threshold value.

In some examples the first set of eye positions 100 and/or the second set of eye positions 100 each comprises a single eye position. If one of the first set of eye positions 100 and the second set of eye positions 100 is the position for a left eye and the other of the first set of eye positions 100 and the second set of eye positions 100 is the position for a right eye, then a mirror procedure can be applied when the eye positions are compared in S106 so as to determine if these eye positions differ from each other less than the threshold value or not.

Embodiments relating to further details of eye position determination of a subject 310 depicted in an image set as performed by the eye tracking system 500 will now be disclosed.

In some aspects, the first eye tracking procedure and the second eye tracking procedure operate independently of each other. In particular, in some embodiments, the second information is obtained independently of the first information.

There may be different examples of pieces of first information and second information. This might also reflect what kind of output the first eye tracking procedure and the second eye tracking procedure output. Aspects relating thereto will be disclosed next.

In some aspects, the first eye tracking procedure outputs glint positions and/or cornea positions. That is, in some examples, the first information pertains to at least one of: glint positions 115 and cornea positions 104 of the subject 310. In some aspects, the second eye tracking procedure outputs head pose and/or pupil positions. That is, in some examples, the second information pertains to at least one of: head pose, comprising the eye position, and pupil positions 101 of the subject 310.

Eye positions 100 given by the glints might then be compared to eye positions 100 given by head pose and/or pupil positions. That is, according to an embodiment, determining (as in S106) whether the first set of eye positions 100, as indicated by the first information, and the second set of eye positions 100, as indicated by the second information, differ from each other less than the threshold value or not involves determining how much the first set of eye positions 100 as determined from the glint positions 115 and/or cornea positions 104 of the subject 310 differ from the first set of eye positions 100 as determined from the head pose and/or pupil positions 101 of the subject 310.

In further aspects, the first eye tracking procedure uses glint positions and/or cornea positions to determine eye positions 100. That is, in some examples, the first set of features pertains to at least one of: glint positions 115 and cornea positions 104 of the subject 310, and the first information is the first set of eye positions 100 itself. In some aspects, the second eye tracking procedure uses head pose and/or pupil positions to determine eye positions 100. That is, in some examples the second set of features pertains to at least one of: head pose and pupil positions 101 of the subject 310, and wherein the second information is the second set of eye positions 100 itself.

Gaze might then be calculated from the first and/or second set of eye positions 100. That is, in some embodiments, the gaze of the subject 310 is calculated using at least one of the first set of eye positions 100 and the second set of eye positions 100.

There could be different ways for the eye tracking system 500 to proceed when the results from the first eye tracking procedure and the second eye tracking procedure are too different from each other. In some aspects, no gaze is calculated when this occurs. That is, in some embodiments, no gaze of the subject 310 in the image set 510 is calculated when the first set of eye positions 100 and the second set of eye positions 100 do not differ from each other less than the threshold value.

In some aspects, the eye positions 100 of the eye tracking procedure associated with the higher level of confidence of the first and second levels of confidence are used, for example when determining the gaze. In particular, according to an embodiment, the first information is associated with a first level of confidence, and the second information is associated with a second level of confidence, and the gaze of the subject 310 is calculated using that of the first set of eye positions 100, as indicated by the first information, and the second set of eye positions 100, as indicated by the second information, associated with the higher level of confidence of the first and second levels of confidence.

There could be different ways to measure the confidence level. In some aspects, the confidence level is binary, which implies that either an eye tracking procedure is used or not used. In particular, in some embodiments each level of confidence takes one of (only) two values, where one of the values defines the eye positions 100 to be accurate and the other of the values defines the eye positions 100 to be inaccurate.

There could be different types of first eye tracking procedures and second eye tracking procedures. In some examples, the first eye tracking procedure is a PCCR based eye tracking procedure. In some examples, the second eye tracking procedure is a non-PCCR based eye tracking procedure. In turn, there could be different examples of non-PCCR based eye tracking procedures. In some examples the non-PCCR based eye tracking procedure is based on tracking head pose and pupil or head pose and iris. From head pose the gaze origin(s), in terms of eye position or eye ball centre or cornea centre, can be calculated as known positions relative to the head. The gaze direction might then be set so that it passes through the pupil as seen in the image capturing unit. In some examples the non-PCCR based eye tracking procedure is based on tracking facial features, including, but not necessarily limited to, iris and pupil, and performing machine learning. Based on data with known gaze angles (as provided by means of known gaze stimulus), a network can be trained to infer gaze from the facial features. In some examples the non-PCCR based eye tracking procedure is based on end-to-end machine learning, where a network is trained based on images with known gaze angles (as provided by means of known gaze stimulus) to infer gaze. In some examples the non-PCCR based eye tracking procedure is based on tracking pupil or iris projection in the image capturing unit. For larger gaze angles from the image capturing unit the projection of the pupil on the sensor of the image capturing unit will be more elliptic than for smaller angles. This gives the gaze direction. Gaze origin, in terms of eye position or eye ball centre or cornea centre, can be calculated from e.g. a known head pose.

The eye tracking system 500 might be part of a vehicle. The subject 310 might then be a driver or a passenger of the vehicle. For example, in case the eye tracking system 600 is part of a vehicle, the gaze, represented by a gaze signal, could be used as input to an advanced driver-assistance system (ADAS), a driver monitoring system (DMS), and/or a driver attention monitor (DAM) system, or the like.

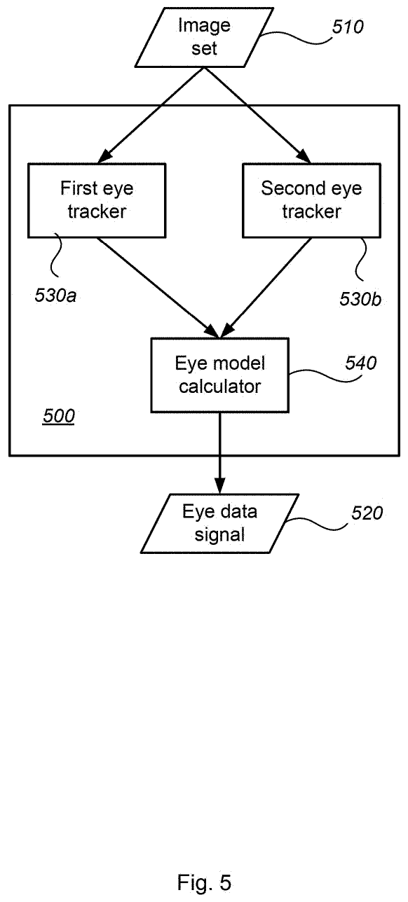

One particular method for eye position determination of a subject 310 depicted in an image set 510 based on at least some of the embodiments disclosed above will now be disclosed with reference to FIG. 5. FIG. 5 schematically illustrates an eye tracking system 500. The eye tracking system 500 operates on images from the image set 510. The eye tracking system 500 comprises a first eye tracker module 530a running the first eye tracking procedure as disclosed above, thus implementing S102. The eye tracking system 500 comprises a second eye tracker module 530b running the second eye tracking procedure as disclosed above, thus implementing S104. The eye tracking system 500 further comprises an eye model calculator module 540 configured to determine eye data of the subject 310 in the image set 510 based on the output from the first eye tracker module 530a and/or the second eye tracker module 530b only when the first set of eye positions 100, as indicated by the output of the first eye tracker module 530a, and the second set of eye positions 100, as indicated by the output from the second eye tracker module 530b, differ from each other less than a threshold value. The output of the eye model calculator module 540 is an eye data signal 520 that, as disclosed above could be used as input to an ADA, a DMS, and/or a DAM system, or the like. The eye data signal 520 may comprise eye position or gaze of the subject 310.

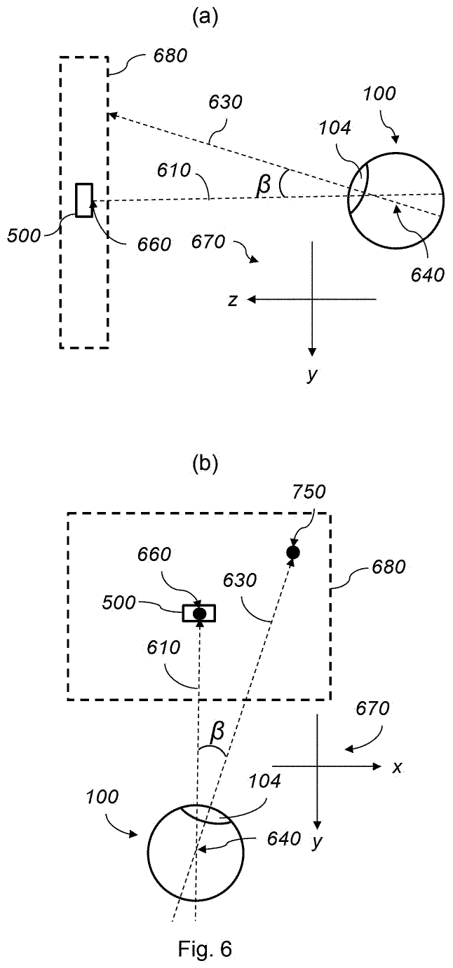

Further aspects of the relation between eye position, gaze origin, gaze direction and gaze point will now be disclosed with reference to FIG. 6 which at (a) and (b) schematically illustrates an eye 100 gazing at a target scene 680 and where the gaze of the eye 100 is tracked by an eye tracking system 500. A gaze angle β is defined as the angle between an axis 610 of the eye tracking system 500 and a gaze direction 630 of the eye 100 of the subject. The axis 610 of the eye tracking system 500 is defined as a vector passing through a focal point 640 of the eye 100 and an origin 660 of coordinates of an internal coordinate system 670 of the eye tracking system 500. The gaze direction 630 of the eye 100 is defined as a vector passing through the focal point 640 of the eye 100 and a gaze point 650 of the eye 100 at the target scene 680. The visual axis 109 of the eye 100, described in relation to FIG. 2, may be referred to as the gaze direction 630. The focal point 640 may be referred to as gaze origin and typically refers to the center of the eye 100, the center of the eye ball of the eye 100, or the center of the cornea 104 of the eye 100. In another example, any point in the eye 100 may be referred to as gaze origin. In yet another example, any point between the eyes 100 of the subject 310 may be referred to as gaze origin.

Although the herein disclosed embodiments have been presented in the context of running a first eye tracking procedure and second eye tracking procedure, the skilled person would understand from this disclosure how to extend the embodiments to a redundant eye tracking system 500 running more than two (independent) eye tracking procedures.

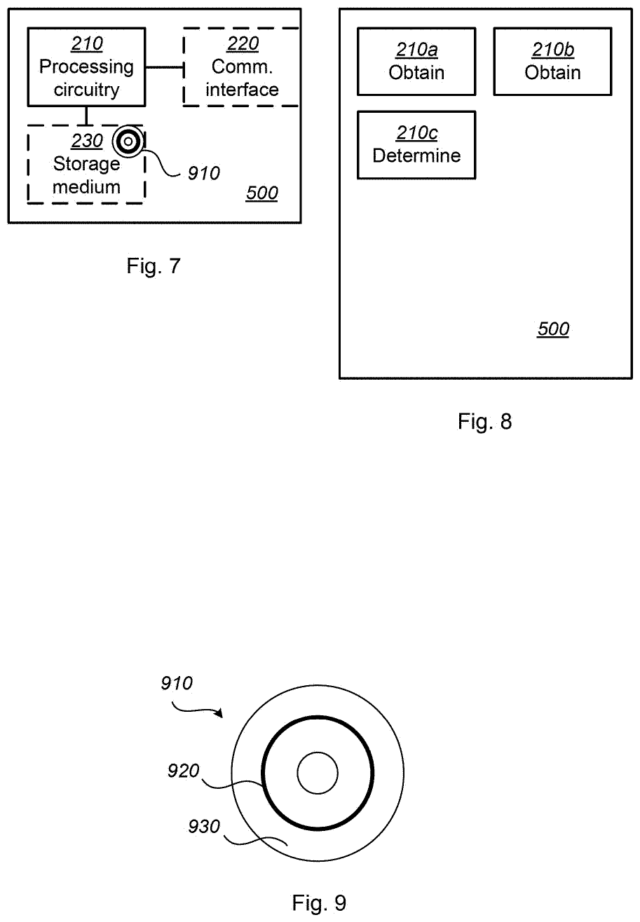

FIG. 7 schematically illustrates, in terms of a number of functional units, the components of an eye tracking system 500 according to an embodiment. Processing circuitry 210 is provided using any combination of one or more of a suitable central processing unit (CPU), multiprocessor, microcontroller, digital signal processor (DSP), etc., capable of executing software instructions stored in a computer program product 910 (as in FIG. 9), e.g. in the form of a storage medium 230. The processing circuitry 210 may further be provided as at least one application specific integrated circuit (ASIC), or field programmable gate array (FPGA).

Particularly, the processing circuitry 210 is configured to cause the eye tracking system 500 to perform a set of operations, or steps, as disclosed above. For example, the storage medium 230 may store the set of operations, and the processing circuitry 210 may be configured to retrieve the set of operations from the storage medium 230 to cause the eye tracking system 500 to perform the set of operations. The set of operations may be provided as a set of executable instructions.

Thus the processing circuitry 210 is thereby arranged to execute methods as herein disclosed. The storage medium 230 may also comprise persistent storage, which, for example, can be any single one or combination of magnetic memory, optical memory, solid state memory or even remotely mounted memory. The eye tracking system 500 may further comprise a communications interface 220 at least configured for communications with other component, functions, nodes, modules, and devices that are operatively connected to the eye tracking system 500. As such the communications interface 220 may comprise one or more transmitters and receivers, comprising analogue and digital components. The processing circuitry 210 controls the general operation of the eye tracking system 500 e.g. by sending data and control signals to the communications interface 220 and the storage medium 230, by receiving data and reports from the communications interface 220, and by retrieving data and instructions from the storage medium 230. Other components, as well as the related functionality, of the eye tracking system 500 are omitted in order not to obscure the concepts presented herein.

In some examples the eye tracking system 500 further comprises one or more image capturing units. The image capturing unit might be an image sensor or a camera, such as a charge-coupled device (CCD) camera or a Complementary Metal Oxide Semiconductor (CMOS) camera. However, other types of image capturing units are also be envisaged.

FIG. 8 schematically illustrates, in terms of a number of functional modules, the components of an eye tracking system 500 according to an embodiment. The eye tracking system 500 of FIG. 8 comprises a number of functional modules; an obtain module 210a configured to perform step S102, an obtain module 21b configured to perform step S104, and a determine module 210c configured to perform step S106. The eye tracking system 500 of FIG. 8 may further comprise a number of optional functional modules. In general terms, each functional module 210a-210c may in one embodiment be implemented only in hardware and in another embodiment with the help of software, i.e., the latter embodiment having computer program instructions stored on the storage medium 230 which when run on the processing circuitry makes the eye tracking system 500 perform the corresponding steps mentioned above in conjunction with FIG. 8. It should also be mentioned that even though the modules correspond to parts of a computer program, they do not need to be separate modules therein, but the way in which they are implemented in software is dependent on the programming language used. Preferably, one or more or all functional modules 210a-210c may be implemented by the processing circuitry 210, possibly in cooperation with the communications interface 220 and/or the storage medium 230. The processing circuitry 210 may thus be configured to from the storage medium 230 fetch instructions as provided by a functional module 210a-210c and to execute these instructions, thereby performing any steps as disclosed herein.

The eye tracking system 500 may be provided as a standalone device or as a part of at least one further device. For example, the eye tracking system 500 might be provided in a vehicle. In particular, according to an embodiment, a vehicle is provided that comprises the eye tracking system 500 as herein disclosed. The vehicle might be a car, a cabin of a truck, etc. The subject 310 might then be a driver or a passenger of the vehicle.

Alternatively, functionality of the eye tracking system 500 may be distributed between at least two devices, or nodes. Thus, a first portion of the instructions performed by the eye tracking system 500 may be executed in a first device, and a second portion of the of the instructions performed by the eye tracking system 500 may be executed in a second device; the herein disclosed embodiments are not limited to any particular number of devices on which the instructions performed by the eye tracking system 500 may be executed. Hence, the methods according to the herein disclosed embodiments are suitable to be performed by an eye tracking system 500 residing in a cloud computational environment. Therefore, although a single processing circuitry 210 is illustrated in FIG. 7 the processing circuitry 210 may be distributed among a plurality of devices, or nodes. The same applies to the functional modules 210a-210c of FIG. 8 and the computer program 920 of FIG. 9.

FIG. 9 shows one example of a computer program product 910 comprising computer readable storage medium 930. On this computer readable storage medium 930, a computer program 920 can be stored, which computer program 920 can cause the processing circuitry 210 and thereto operatively coupled entities and devices, such as the communications interface 220 and the storage medium 230, to execute methods according to embodiments described herein. The computer program 920 and/or computer program product 910 may thus provide means for performing any steps as herein disclosed.

In the example of FIG. 9, the computer program product 910 is illustrated as an optical disc, such as a CD (compact disc) or a DVD (digital versatile disc) or a Blu-Ray disc. The computer program product 910 could also be embodied as a memory, such as a random access memory (RAM), a read-only memory (ROM), an erasable programmable read-only memory (EPROM), or an electrically erasable programmable read-only memory (EEPROM) and more particularly as a non-volatile storage medium of a device in an external memory such as a USB (Universal Serial Bus) memory or a Flash memory, such as a compact Flash memory. Thus, while the computer program 920 is here schematically shown as a track on the depicted optical disk, the computer program 920 can be stored in any way which is suitable for the computer program product 910.

The inventive concept has mainly been described above with reference to a few embodiments. However, as is readily appreciated by a person skilled in the art, other embodiments than the ones disclosed above are equally possible within the scope of the inventive concept, as defined by the appended patent claims.

1. An eye tracking system for eye position determination of a subject depicted in an image set, the eye tracking system being configured to:
obtain first information indicating a first set of eye positions of the subject by applying a first eye tracking procedure on an image set depicting the subject, the first eye tracking procedure using a first set of features extracted from the image set for obtaining the first set of eye positions;
obtain second information indicating a second set of eye positions of the subject by applying a second eye tracking procedure on the image set depicting the subject, the second eye tracking procedure using a second set of features extracted from the image set for obtaining the second set of eye positions; and
determine the eye position of the subject in the image set based on the first information and the second information only when the first set of eye positions, as indicated by the first information, and the second set of eye positions, as indicated by the second information, differ from each other less than a threshold value.

2. The eye tracking system according to claim 1, wherein the first information pertains to at least one of: glint positions and cornea positions of the subject.

3. The eye tracking system according to claim 1, wherein the second information pertains to at least one of: head pose, comprising the eye position, and pupil positions of the subject.

4. The eye tracking system according to claim 2, wherein the second information pertains to at least one of: head pose, comprising the eye position, and pupil positions of the subject and; wherein determining whether the first set of eye positions, as indicated by the first information, and the second set of eye positions, as indicated by the second information, differ from each other less than the threshold value or not involves determining how much the first set of eye positions as determined from the glint positions and/or cornea positions of the subject differ from the second set of eye positions as determined from the head pose and/or pupil positions of the subject.

5. The eye tracking system according to claim 1, wherein the first set of features pertains to at least one of: glint positions and cornea positions of the subject, and wherein the first information is the first set of eye positions itself.

6. The eye tracking system according to claim 1, wherein the second set of features pertains to at least one of: head pose and pupil positions of the subject, and wherein the second information is the second set of eye positions itself.

7. The eye tracking system according to claim 5 wherein the second set of features pertains to at least one of: head pose and pupil positions of the subject, and wherein the second information is the second set of eye positions itself and, wherein gaze of the subject is calculated using at least one of the first set of eye positions and the second set of eye positions.

8. The eye tracking system according to claim 1, wherein no gaze of the subject in the image set is calculated when the first set of eye positions and the second set of eye positions do not differ from each other less than the threshold value.

9. The eye tracking system according to claim 1, wherein the second information is obtained independently of the first information.

10. The eye tracking system according to claim 1, wherein the first information is associated with a first level of confidence, wherein the second information is associated with a second level of confidence, and wherein gaze of the subject is calculated using that of the first set of eye positions, as indicated by the first information, and the second set of eye positions, as indicated by the second information, associated with the higher level of confidence of the first and second levels of confidence.

11. The eye tracking system according to claim 10, wherein each level of confidence takes one of two values, wherein one of the values defines the eye positions to be accurate and the other of the values defines the eye positions to be inaccurate.

12. The eye tracking system according to claim 1, wherein the first eye tracking procedure is a PCCR based eye tracking procedure.

13. The eye tracking system according to claim 1, wherein the second eye tracking procedure is a non-PCCR based eye tracking procedure.

14. A vehicle comprising the system according to claim 1, wherein the subject is a driver or a passenger of the vehicle.

15. A method for eye position determination of a subject depicted in an image set, the method comprising:
obtaining first information indicating a first set of eye positions of the subject by applying a first eye tracking procedure on an image set depicting the subject, the first eye tracking procedure using a first set of features extracted from the image set for obtaining the first set of eye positions;
obtaining second information indicating a second set of eye positions of the subject by applying a second eye tracking procedure on the image set depicting the subject, the second eye tracking procedure using a second set of features extracted from the image set for obtaining the second set of eye positions; and
determining the eye position of the subject in the image set based on the first information and the second information only when the first set of eye positions, as indicated by the first information, and the second set of eye positions, as indicated by the second information, differ from each other less than a threshold value.

16. A computer program for eye position determination of a subject depicted in an image set, the computer program comprising computer code which, when run on processing circuitry of an eye tracking system, causes the eye tracking system to:
obtain first information indicating a first set of eye positions of the subject by applying a first eye tracking procedure on an image set depicting the subject, the first eye tracking procedure using a first set of features extracted from the image set for obtaining the first set of eye positions;
obtain second information indicating a second set of eye positions of the subject by applying a second eye tracking procedure on the image set depicting the subject, the second eye tracking procedure using a second set of features extracted from the image set for obtaining the second set of eye positions; and
determine the eye position of the subject in the image set based on the first information and the second information only when the first set of eye positions, as indicated by the first information, and the second set of eye positions, as indicated by the second information, differ from each other less than a threshold value.

17. A computer program product comprising a computer program according to claim 16, and a computer readable storage medium on which the computer program is stored.
